(19) United States
(12) Patent Application Publication
(10) Pub. No.: US 2023/0099941 A1
(43) Pub. Date: Mar. 30, 2023

(54) BAD-ODOR CONTROL USING ENCAPSULATION TECHNOLOGIES CONTAINING A NEUTRALIZING AGENT

(71) Applicant: Henkel AG & Co. KGaA, Duesseldorf (DE)

(72) Inventors: Andreas BAUER, Kaarst (DE); Marc WEYHE, Krefeld (DE); Erwan GUILMET, Duesseldorf (DE); Rainer SORG, Kempen (DE); Madeline SCHEUER, Moers (DE)

(21) Appl. No.: 17/799,692

(22) PCT Filed: Feb. 4, 2021
(86) PCT No.: PCT/EP2021/052621

§ 371 Date: Aug. 15, 2022

(30) Foreign Application Priority Data
Feb. 19, 2020 (EP) 20158274.9

(51) Int. Cl. C11D 3/50; C11D 3/00; C11D 3/28; C11D 3/20; C11D 3/37; C11D 11/00; C11D 17/04; C11D 17/00

(52) U.S. Cl. CPC C11D 3/505; C11D 3/0015; C11D 3/28; C11D 3/2086; C11D 3/3738; C11D 11/0017; C11D 17/046; C11D 17/0013

(57) ABSTRACT

A composition may include microcapsules containing at least one odor-neutralizing agent, and agents may include such a composition. Furthermore, such compositions or agents may reduce malodors and/or be used for treating a surface, for treating room air, or for washing and/or caring for textiles using such compositions or agents.

### CROSS-REFERENCE TO RELATED APPLICATION

The present application is a national stage entry according to 35 U.S.C. § 371 of PCT application No.: PCT/EP2021/052621 filed on Feb. 4, 2021; which claims priority to European patent application 20158274.9, filed on Feb. 19, 2020; all of which are incorporated herein by reference in their entirety and for all purposes.

### TECHNICAL FIELD

The present disclosure relates to a composition comprising microcapsules containing at least one odor-neutralizing agent, and to agents containing such a composition. Furthermore, the present disclosure is directed to the use of such compositions or agents to reduce malodors and to methods for treating a surface, for treating room air, or for washing and/or caring for textiles using such compositions or agents.

### BACKGROUND

Commonly used washing and cleaning agents as well as cosmetic products are usually perfumed. Consumers desire both a sufficient intrinsic scent of the product as well as a reduction of “bad” secondary odors. Furthermore, long-lasting and fresh perfumes are desired. Bad smells or malodors come from certain odor-active compounds, such as certain ketones or aldehydes, e.g., 2,4-decadienal, in particular short-chain aldehydes.

### SUMMARY

The consumer may have the possibility of bringing about a reduction in malodors, in particular those which can be attributed to aldehydes and/or ketones.

It has now been found that this objective is achieved by formulations containing encapsulated odor-neutralizing agents.

In a first aspect, a composition may include microcapsules comprising at least one core and one shell, the core containing at least one odor-neutralizing agent, preferably selected from:

a) a 2-amino-1,3-propanediol compound of the general formula (I)

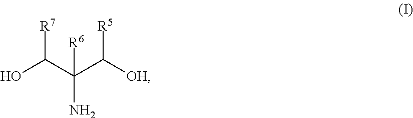

wherein, in the compounds according to formula (I), R⁵, R⁶ and R⁷, in each case independently of one another, represent H or hydrocarbon groups;
   b) a 1-aza-3,7-dioxabicyclo[3.3.0]octane compound of the general formula (II)

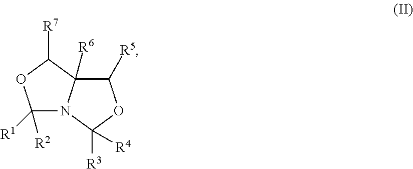

wherein, in the compounds according to formula (II), R¹, R², R³ and R⁴, in each case independently of one another, represent groups which, in a compound of the general formula R¹—C(═O)—R² or R³—C(═O)—R⁴, produce a fragrance aldehyde having at least 6 carbon atoms or a fragrance ketone having at least 6 carbon atoms, and R⁵, R⁶ and R⁷, in each case independently of one another, represent H or a hydrocarbon group which can be acyclic or cyclic, substituted or unsubstituted, branched or unbranched, saturated or unsaturated; or
   c) combinations of compounds a) and b).

In a further aspect, an agent may include at least one composition as described herein, the agent being a washing agent, cleaning agent or treatment agent.

In yet another aspect, a composition described herein or agent described herein may reduce malodors.

Finally, in a final aspect, a method for treating a surface, for treating room air, or for washing and/or caring for textiles is disclosed, characterized in that a composition described herein and/or an agent described herein is used in at least one method step.

These and other aspects, features, and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

### DETAILED DESCRIPTION

Unless indicated otherwise, all percentages indicated are percentages by weight, in each case based on the total weight of the corresponding composition. Numerical ranges that are indicated in the format “from x to y” also include the stated values. If several preferred numerical ranges are indicated in this format, it is readily understood that all ranges that result from the combination of the various endpoints are also included.

“At least one,” as used herein, refers to 1 or more, for example 1, 2, 3, 4, 5, 6, 7, 8, 9 or more.

“Approximately,” as used herein in relation to numerical values, refers to the corresponding value ±10%, preferably ±5%.

As was surprisingly found, the reduction, i.e., in the context of the neutralization, of bad odors or malodors, i.e., unwanted odors, by the below-described encapsulated odor-neutralizing agents in the end product, i.e., for example in a universal washing agent, in comparison with end products having odor-neutralizing agents, albeit in non-encapsulated form, can be significantly increased. The improvement is expressed not only by increased (direct) malodor neutralization, as defined below, but also by improved longevity of the malodor neutralization. In particular, it has been found that the neutralization of malodors that are attributable to ketones and/or aldehydes, in particular short-chain aldehydes, can be improved.

Neutralizing malodors means not only masking malodors, but deactivating the malodor-triggering substances. Deactivation means here that the malodor is at least reduced and in particular even completely removed, i.e., eliminated. Whenever a malodor is present, there must be an object from which that malodor emanates, or a room or system in which the malodor is perceptible. A composition can be applied to this object or room, for example as an additive/constituent of a washing agent, cleaning agent or treatment agent, as defined herein.

Accordingly, a composition may include microcapsules comprising at least one core and one shell, the core containing at least one odor-neutralizing agent.

Odor-neutralizing agents which are suitable for use are known in principle in the prior art and include all those compounds which bring about, for example when they adhere to a piece of fabric, the neutralization of malodors as defined above.

Suitable odor-neutralizing agents that are particularly preferred, are selected from the group consisting of

a) 2-amino-1,3-propanediol compounds of the general formula (I)

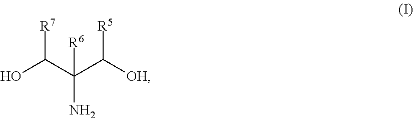

wherein, in the compounds according to formula (I), R⁵, R⁶ and R⁷, in each case independently of one another, represent H or hydrocarbon groups;
b) 1-aza-3,7-dioxabicyclo[3.3.0]octane compounds of the general formula (II)

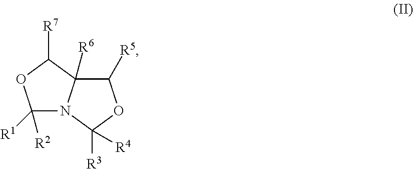

wherein, in the compounds according to formula (II), R¹, R², R³ and R⁴, in each case independently of one another, represent groups which, in a compound of the general formula R¹—C(═O)—R² or R³—C(═O)—R⁴, produce a fragrance aldehyde having at least 6 carbon atoms or a fragrance ketone having at least 6 carbon atoms, and R⁵, R⁶ and R⁷, in each case independently of one another, represent H or a hydrocarbon group which can be acyclic or cyclic, substituted or unsubstituted, branched or unbranched, saturated or unsaturated; and
c) combinations of compounds a) and b).

It is believed that the amino alcohols of formula (I), as well as the amino alcohols released by hydrolysis of the compound of formula (II), cause malodorous substances to be deactivated. The odorant aldehydes and/or odorant ketones likewise released during the hydrolysis of the compounds of formula (II) also contribute to masking the malodor. A significant reduction up to the elimination of malodors is therefore made possible.

In principle, all hydrocarbon groups can be acyclic or cyclic, substituted or unsubstituted, branched or unbranched and saturated or unsaturated. The hydrocarbon groups can in principle be heteroatoms, such as nitrogen atoms, oxygen atoms or sulfur atoms. Preferred with regard to R⁵, R⁶ and R⁷ are in each case acyclic, unbranched hydrocarbon groups, which can optionally be substituted. Suitable substituents are, for example, hydroxy groups, alkoxy groups, amino groups or halogen groups. The hydrocarbon group can be, for example, linear or branched, substituted or unsubstituted alkyl having 1 to 10 carbon atoms, for example having 1 to 6 carbon atoms or 1, 2, 3, 4, 5 or 6 carbon atoms, The corresponding alkoxy groups or hydroxy groups, amino groups or haloalkyl groups can also be used as substituted variants.

The amino alcohol compounds of formula (I) are 2-amino-1,3-propanediol compounds. R⁶ can be hydrogen or alkyl, which can be substituted by one or two hydroxyl groups and/or an amino group, it being possible to replace up to 8 non-adjacent —CH₂ groups with —O—. Alkyl groups are preferably C₁₋₂₄ alkyl groups, particularly preferably C₁₋₁₆ alkyl groups, in particular C₁₋₁₂ alkyl groups, especially C₁₋₆ alkyl groups, by way of example C₁₋₃ alkyl groups. Alkyl groups can be linear, branched or cyclic. They are preferably linear alkyl groups. They can be mono- or dihydroxyalkyl groups, which can also have an amino group instead of or in addition to the hydroxyl groups. The alkyl groups can also be substituted or unsubstituted. If the alkyl groups are interrupted by —O—, they are preferably structural elements of the formula —CH₂—CH₂—O— or —CH₂—CH(CH₃)—O—. Such compounds are easily obtainable by alkoxylation of the corresponding hydroxy compounds.

Particularly preferred groups R⁶ in formula (I) are methyl groups, ethyl groups and hydroxymethyl groups.

R⁵ and R⁷ are hydrogen or a Ci-s alkyl group, which can optionally be substituted, preferably a C₁₋₃ alkyl group. R⁵ and R⁷ are particularly preferably hydrogen or a methyl or ethyl group, in particular hydrogen.

Preferred amino alcohol compounds are 2-amino-1,3-propanediol, 2-amino-2-hydroxymethyl-1,3-propanediol, 2-amino-2-methyl-1,3-propanediol, 2-amino-2-ethyl-1,3-propanediol and mixtures thereof.

As already defined, in formula (II), R¹, R², R³, R⁴, in each case independently of one another, represent groups which, in a compound of the general formula R¹—C(═O)—R² or R³—C(═O)—R⁴, produce an odorant aldehyde having at least 6 carbon atoms or an odorant ketone having at least 6 carbon atoms.

Preferably, in one of the structural elements —CR¹R² or —CR³R⁴, there are at most R¹ and R² or R³ and R⁴ which, in a compound of the general formula R¹—C(═O)—R² or R³—C(═O)—R⁴, produce an odorant ketone. In particular, in the two structural elements —CR¹R² and —CR³R⁴, there are R¹ and R² and R³ and R⁴ which, in a compound of the general formula R¹—C(═O)—R² and R³—C(═O)—R⁴, each produce an odorant aldehyde, in particular the same odorant aldehyde. Suitable odorant ketones and aldehydes are known to a person skilled in the art and are listed below by way of example.

According to a preferred embodiment, the odorant aldehyde is selected from adoxal (2,6,10-trimethyl-9-undecenal), anisaldehyde (4-methoxybenzaldehyde), cymene (3-(4-isopropyl-phenyl)-2-methylpropanal), ethylvanillin, Florhydral (3-(3-isopropylphenyl)butanal), Helional (3-(3,4-methylenedioxyphenyl)-2-methylpropanal), heliotropin, hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenealdehyde, vanillin, 2,6,10-trimethyl-9-undecenal, 3-dodecen-1-al, alpha-n-amylcinnamaldehyde, melonal (2,6-dimethyl-5-heptenal), 2,4-di-methyl-3-cyclohexene-1-carboxaldehyde (Triplal), 4-methoxybenzaldehyde, benzaldehyde, 3-(4-tert-butylphenyl)-propanal, 2-methyl-3-(para-methoxyphenyl)propanal, 2-methyl-4-(2,6,6-timethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, 3-ethoxy-4-hydroxybenzaldehyde, para-ethyl-alpha,alpha-dimethylhydrocinnamaldehyde, alpha-methyl-3,4-(methylenedioxy)-hydrocinnamaldehyde, 3,4-methylenedioxybenzaldehyde, alpha-n-hexylcinnamaldehyde, m-cymene-7-carboxaldehyde, alpha-methylphenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexene carboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methyl-undecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanindan-1- or 2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexene-carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanindan-1-carboxaldehyde, 2-methyloctanal, alpha-methyl-4-(1-methylethyl)benzeneacetaldehyde, 6,6-dimethyl-2-norpinene-2-propionaldehyde, para-methylphenoxyacetaldehyde, 2-methyl-3-phenyl-2-propen-1-al, 3,5,5-trimethylhexanal, hexahydro-8,8-dimethyl-2-naphthaldehyde, 3-propyl-bicyclo-[2.2.1]-hept-5-ene-2-carbaldehyde, 9-decenal, 3-methyl-5-phenyl-1-pentanal, methylnonylacetaldehyde, hexanal and trans-2-hexenal.

According to a preferred embodiment, the odorant ketone is selected from methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, Iso-E-Super (1-(1,2,3,4,5,6,7,8-octahydro-2,3,8,8-tetramethyl-2-naphthalenyl)-ethan-1-one (and isomers)), methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyldecahydro-2-naphthone, dimethyloctenone, frescomenthe (2-butan-2-yl-cyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menthen-6(2)-yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl (4-(1,3-benzodioxol-5-yl)butan-2-one), Hexalone (1-(2,6,6-trimethyl-2-cyclohexen-1-yl)-1,6-heptadien-3-one), Isocyclemone E (2-acetonaphthone-1,2,3,4,5,6,7,8-octahydro-2,3,8,8-tetramethyl), methyl nonylketone, methylcyclocitrone, methyl lavender ketone, Orivone (4-tert-amyl-cyclohexanone), 4-tert-butylcyclohexanone, Delphone (2-pentyl-cyclopentanone), muscone (CAS 541-91-3), Neobutenone (1-(5,5-dimethyl-1-cyclohexenyl)pent-4-en-1-one), plicatone (CAS 41724-19-0), Veloutone (2,2,5-trimethyl-5-pentylcyclopentan-1-one), 2,4,4,7-tetramethyl-oct-6-en-3-one and tetramerane (6,10-dimethylundecen-2-one).

According to a preferred embodiment, R⁵ and R⁷, in each case independently of one another, advantageously each represent hydrogen or a C₁₋₆ hydrocarbon group which can optionally be substituted, preferably a C₁₋₃ hydrocarbon group. R⁵ and R⁷ are particularly preferably each hydrogen or each a methyl or ethyl group, but in particular each hydrogen.

According to a preferred embodiment, R¹ and R³ are, independently of one another, advantageously each a C₆₋₂₄ hydrocarbon group, preferably a C₇₋₂₄ hydrocarbon group, it being possible for the hydrocarbon group to be acyclic or cyclic, substituted or unsubstituted, branched or unbranched and saturated or unsaturated.

In another preferred embodiment, R², R⁴, R⁵, R⁷ are each hydrogen, R¹ and R³ are each a C₆₋₂₄ hydrocarbon group, preferably a C₇₋₂₄ hydrocarbon group, it being possible for the hydrocarbon group to be acyclic or cyclic, substituted or unsubstituted, branched or unbranched and saturated or unsaturated, and R⁶ represents hydrogen or a C₁₋₂₄ hydrocarbon group which can be acyclic or cyclic, substituted or unsubstituted, branched or unbranched and saturated or unsaturated and which can be substituted in particular with one or two hydroxyl groups and/or an amino group, it being possible to replace up to 8 non-adjacent —CH₂ groups with —O—.

In relation to R⁶, preferred groups are C₁₋₁₆ hydrocarbon groups, in particular C₁₋₁₂ hydrocarbon groups, more preferably Ci-s hydrocarbon groups, most preferably C₁₋₃ hydrocarbon groups. They are preferably unbranched, acyclic alkyl groups. They can also be substituted. They can be, for example, mono- or dihydroxyalkyl groups, which can also have an amino group instead of or in addition to the hydroxyl groups. If the hydrocarbon groups are interrupted by —O—, they are preferably structural elements of the formula —CH₂—CH₂—O— or —CH₂—CH(CH₃)—O—. Such compounds are easily obtainable by alkoxylation of the corresponding hydroxy compounds. Very particularly preferred groups R⁶ are methyl, ethyl or hydroxymethyl groups or hydrogen.

A preferred embodiment is present when R² and R⁴ each represent hydrogen in the 1-aza-3,7-dioxabicyclo[3.3.0]octane compound of the general formula (II).

In further preferred compounds of the general formula (II), R², R⁴, R⁵, R⁶, R⁷ are each hydrogen and R¹ and R³ are each a C₅₋₂₄ hydrocarbon group. (EP2809756)

Another preferred embodiment is present when, in the 1-aza-3,7-dioxabicyclo[3.3.0]octane compound of the general formula (II), R², R⁴, R⁵, R⁷ each represent hydrogen, and R⁶ represents a methyl, ethyl or hydroxymethyl group or hydrogen, and R¹ and R³, independently of one another, each represent a C₆₋₂₄ hydrocarbon group, preferably a C₇₋₂₄ hydrocarbon group, it being possible for the hydrocarbon group to be acyclic or cyclic, substituted or unsubstituted, branched or unbranched and saturated or unsaturated.

Accordingly, suitable oxazolidines of the general formula (II) are, for example, 1-aza-3,7-dioxa-2,8-diheptylbicyclo[3.3.0]octane, 1-aza-3,7-dioxa-2,8-diheptyl-5-methyl-bicyclo[3.3.0]octane, 1-aza-3,7-dioxa-2,8-diheptyl-5-hydroxymethyl-bicyclo[3.3.0]octane, 1-aza-3,7-dioxa-2,8-diheptyl-5-ethylbicyclo[3.3.0]octane,1-aza-3,7-dioxa-2,8-dioctylbicyclo[3.3.0]octane, 1-aza-3,7-dioxa-2,8-dioctyl-5-methyl-bicyclo[3.3.0]octane, 1-aza-3,7-dioxa-2,8-dioctyl-5-hydroxymethyl-bicyclo[3.3.0]octane and 1-aza-3,7-dioxa-2,8-dioctyl-5-ethylbicyclo[3.3.0]octane.

The compounds of the general formula (II) are obtainable in particular by reacting compounds of the general formula (I)

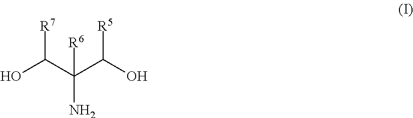

with compounds of the general formulas R¹—C(═O)—R² and R³—C(═O)—R⁴ with ring closure. A suitable substance according to formula (I) is, for example, 2-aminopropane-1,3-diol. What has already been said above applies in each case to R¹ to R⁷. The general formulas R¹—C(═O)—R² and R³—C(═O)—R⁴ very generally represent odorant aldehydes or odorant ketones. Odorant aldehydes are those odorants which are chemically an aldehyde and which advantageously trigger a particularly pleasant olfactory sensation in humans. Odorant ketones are those odorants which are chemically a ketone and which advantageously trigger a particularly pleasant olfactory sensation in humans. Particularly suitable odorant aldehydes and odorant ketones are listed above as examples. Two examples are given for illustration. For example, in the odorant aldehyde octanal corresponding to the general formula R¹—C(═O)—R², R¹ represents a heptyl group (i.e., CH₃—(CH₂)₆)— and R² represents hydrogen or vice versa. For example, in the odorant ketone methyl nonyl ketone according to the general formula R¹—C(═O)—R², R¹ represents a methyl group and R² represents a nonyl group (i.e., CH₃—(CH₂)₈—) or vice versa.

As odorant aldehydes and/or odorant ketones, it is possible in principle to use all customary odorant aldehydes and/or odorant ketones which are used in particular to bring about a pleasant olfactory sensation in humans. Such odorant aldehydes and/or odorant ketones are known to a person skilled in the art and are also described in the patent literature, for example in US 2003/0158079 A1, paragraphs [0154] and [0155].

To prepare the compounds of the general formula (II), a compound of the general formula (I) can therefore be reacted with aldehydes, ketones or mixtures of ketones and aldehydes with ring closure. According to some embodiments, the compounds of the general formula (II) are derived from a molecule of the general formula (I) and two aldehyde molecules, which may be the same or different, or one aldehyde molecule and one ketone molecule. In the reaction of less than stoichiometric amounts of aldehydes and/or ketones, monocyclic compounds are also present in the product mixture. However, the proportion of bicyclic compounds to monocyclic compounds can be adjusted in a simple manner by selecting the molar ratio between aldehyde/ketone and the compound of the general formula (I).

The reaction is preferably carried out in a suitable solvent or in situ. Suitable solvents are, for example, aromatic hydrocarbons such as toluene. The reaction is preferably carried out at a temperature in the range of from 80 to 150° C., particularly preferably 100 to 140° C. For example, the compound of the general formula (I) is placed in the solvent together with the desired ketone and/or aldehyde under a nitrogen atmosphere. The reaction mixture is then heated. Frequently, the water separator is then heated under reflux. The reaction product obtained is isolated by conventional methods and optionally purified. In WO2007/087977 A1, to which reference is hereby made, the preparation of compounds of the general formula (II) is also described in detail using synthesis examples.

In various embodiments, the at least one odor-neutralizing agent, as defined above, can be used in combination with one or more compounds of the general formula (IV)

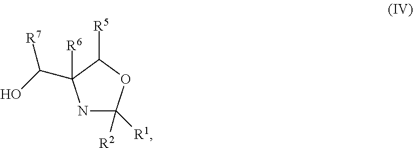

wherein, in the compounds of the general formula (IV),
R¹, R² represent, independently of one another, groups which, in a compound of the general formula R¹—C(═O)—R², produce a fragrance aldehyde having at least 6 carbon atoms or a fragrance ketone having at least 6 carbon atoms, and R⁵, R⁶, R⁷ represent, independently of one another, H or a hydrocarbon group which can be acyclic or cyclic, substituted or unsubstituted, branched or unbranched and saturated or unsaturated.

In various embodiments, the at least one odor-neutralizing agent, i.e., at least one 1-aza-3,7-dioxabicyclo[3.3.0]octane compound of the general formula (II) and/or at least one 2-amino-1,3-propanediol compound of the general formula (I) are used together with odorants, with the 1-aza-3,7-dioxabicyclo[3.3.0]octane compound in amounts by weight, based on the total amount of odorant, preferably being used in the range of from 1:100 to 100:1, in particular from 10:1 to 1:50, for example in the range of from 10:1 to 1:10 or 10:1 to 1:2 or 5:1 to 1:1.

In various embodiments, the odor-neutralizing agent is contained in the capsule with substantially no additional free perfume or fragrance. If the odor-neutralizing agent is a compound of formula (II), the microcapsule may contain, in addition to the compounds of formula (II), the corresponding release products, i.e., the released fragrance and the corresponding compound of formula (I). These can be produced spontaneously. In such embodiments, too, it can be advantageous for the microcapsule to contain no fragrance other than that released from the compounds of formula (II).

In various embodiments, the at least one odor-neutralizing agent is present in the composition in an amount of from approximately 0.1 to approximately 40 wt. %, preferably approximately 0.1 to approximately 20 wt. %, based on the total weight of said composition.

The odor-neutralizing agent is encapsulated in microcapsules in the composition.

“Microcapsule,” as used herein, refers to capsules having a core-shell morphology on a micrometer scale, comprising a capsule shell which completely encloses a core. The odor-neutralizing agent is a constituent of the microcapsule core. The weight ratio between shell and core is usually in the range of from 5:95 to 30:70, preferably in the range of from 10:90 to 20:80, for example 15:85. The “core” here includes the entire content enclosed by the shell, i.e., the odor-neutralizing agent and any additional constituents.

“Completely encloses” or “completely surrounds,” as used herein with reference to the microcapsules, means that the core is completely surrounded by the shell, i.e., it is in particular not embedded in a matrix such that it is exposed at any point. It is also preferable for the capsule shell to be such that the release of the contents is controlled, i.e., the contents are not released in a spontaneous and uncontrolled manner, independently of any release stimulus. For this reason, the capsule shell is preferably substantially impermeable to the encapsulated contents. “Substantially impermeable,” as used in this context, means that the contents of the capsule or individual ingredients cannot spontaneously pass through the shell, but rather the contents can only be released by the capsule being opened or optionally by means of a diffusion process that takes place over a long period of time. The core may be solid, liquid and/or gaseous, but is preferably solid and/or liquid. The microcapsules are preferably substantially spherical and have a diameter in the range of from 0.01 to 1,000 μm, in particular from 0.1 to 500 μm. The capsule shell and capsule core are made of different materials; in particular, under standard conditions (20° C., 1,013 mbar), the capsule shell is preferably solid, and the core is preferably solid and/or liquid, in particular liquid.

High-molecular compounds of animal or vegetable origin, e.g., protein compounds (gelatin, albumin, casein), cellulose derivatives (methylcellulose, ethylcellulose, cellulose acetate, cellulose nitrate, carboxymethylcellulose), and synthetic polymers (e.g., polyamides, polyolefins, polyesters, polyurethanes, epoxy resins, silicone resins and condensation products of carbonyl- and NH group-containing compounds), for example, can very generally be used as the capsule material for the microcapsules. Specifically, the shell material may be selected, for example, from: polyacrylates; polyethylene; polyamides; polystyrenes; polyisoprenes; polycarbonates; polyesters; polyureas; polyurethanes; polyolefins; polysaccharides; epoxy resins; vinyl polymers; urea cross-linked with formaldehyde or glutaraldehyde; melamine cross-linked with formaldehyde; gelatin-polyphosphate coacervates, optionally cross-linked with glutaraldehyde; gelatin-gum arabic coacervates; silicone resins; polyamines reacted with polyisocyanates; acrylate monomers polymerized by means of free radical polymerization; silk; wool; gelatin; cellulose; proteins; and mixtures and copolymers thereof. Polyacrylates, polyethylene, polyamides, polystyrenes, polyisoprenes, polycarbonates, polyesters, polyureas, polyurethanes, polyolefins, epoxy resins, vinyl polymers and urea and/or melamine cross-linked with formaldehyde or glutaraldehyde are particularly preferred. In various embodiments it may be preferable for the encapsulation material to be biodegradable.

Methods which are in principle suitable for producing suitable microcapsules are those known microencapsulation methods in which, for example, the phase to be encapsulated is encapsulated by being coated with film-forming polymers (such as those mentioned above) which precipitate on the material to be covered after emulsification and coacervation or interfacial polymerization.

The phase to be encapsulated comprises at least one odor-neutralizing agent as defined above. Furthermore, according to some embodiments, the phase to be encapsulated can also be a benefit agent composition which comprises at least one odor-neutralizing agent and advantageously contains at least one further benefit agent, for example a perfume oil composition as defined below.

The capsules can release the encapsulated benefit agents using various mechanisms. For example, capsules can be used which have a mechanically stable capsule shell which then becomes permeable to the agents contained therein due to one or more environmental influences, such as changes in the temperature or the ionic strength or the pH of the surrounding medium. Stable capsule wall materials through which the at least one benefit agent, i.e., the at least one odor-neutralizing agent, and optionally further benefit agents, can diffuse over time are also possible. The capsules may release the at least one contained benefit agent preferably when the pH or the ionic strength of the environment changes, when the temperature changes, upon exposure to light, by diffusion and/or under mechanical stress.

In a preferred embodiment, the capsules are fragile, that is to say they can release the encapsulated agent due to mechanical stress, such as friction, pressure, or shear stress, which breaks the shell of the capsules. In another embodiment, the capsule is thermally labile, that is to say encapsulated substances may be released when the capsules are exposed to a temperature of at least 70° C., preferably at least 60° C., more preferably at least 50° C., and in particular at least 40° C.

In another preferred embodiment, the capsule for the encapsulated benefit agent(s) may become permeable after exposure to radiation of a certain wavelength, preferably by exposure to sunlight.

It is also possible that the capsules are fragile and at the same time thermally labile and/or unstable to radiation of a certain wavelength.

Suitable microcapsules can be water-soluble and/or water-insoluble, but are preferably water-insoluble capsules. The water-insolubility of the capsules has the advantage that they can withstand washing, cleaning or other treatment applications and can thus dispense the at least one benefit agent only after the aqueous washing, cleaning or treatment process, such as when drying, by means of a mere increase in temperature or due to sunlight or in particular friction on the surface.

In some embodiments, water-insoluble capsules which are broken up by friction are particularly preferred.

The term “abradable” capsules or capsules that “can be broken up by friction” means in particular those capsules which, when they adhere to a surface treated therewith (e.g., a textile surface), can be opened or broken by mechanical friction or pressure, so that the contents are released only as a result of mechanical action, e.g., if someone dries their hands on a towel on which such capsules are deposited.

Advantageously usable, abradable capsules can have average diameters d₅₀ of <250 μm, preferably in the range of from 1 to 100 μm, more preferably between 3 and 95 μm, in particular between 4 and 90 μm, for example between 5 and 80 μm, for example between 5 and 40 μm. The d₅₀ value indicates the diameter which results when 50 wt. % of the capsules have a smaller diameter and 50 wt. % of the capsules have a larger diameter than the stated d₅₀ value. It is furthermore preferred for the d₉₀ value of the particle size distribution of the microcapsules to be <70 μm, preferably <60 μm, particularly preferably <50 m. The d₉₀ value of the particle size distribution is the value at which 90% of all particles are smaller and 10% of the particles are larger than this value.

The shell of the capsules enclosing the core or (filled) cavity preferably has an average thickness in the range between approximately 50 and 500 nm, preferably between approximately 100 nm and approximately 250 nm. Capsules are particularly abradable if they are within the ranges given above for the average diameter and the average thickness.

The d₅₀ value indicates the diameter which results when 50 wt. % of the capsules have a smaller diameter and 50 wt. % of the capsules have a larger diameter than the stated d₅₀ value. It is furthermore preferred for the d₉₀ value of the particle size distribution of the microcapsules to be <70 μm, preferably <60 μm, particularly preferably <50 m. The d₉₀ value of the particle size distribution is the value at which 90% of all particles are smaller and 10% of the particles are larger than this value.

The diameter of the capsules or the particle size of the microcapsules can be determined by conventional methods. It can be determined, for example, by means of dynamic light scattering, which can usually be carried out on dilute suspensions containing e.g., 0.01 to 1 wt. % of capsules. It can also be determined by evaluating light microscopic or electron microscopic images of capsules.

In various embodiments, a microcapsule has an average diameter d₅₀ of from approximately 1 to 80 μm, preferably approximately 5 to 40 μm, in particular approximately 20 to 35 μm, for example approximately 22 to approximately 33 μm.

The wall material of the microcapsules preferably comprises the above-mentioned compounds, e.g., polyurethanes, polyolefins, polyamides, polyesters, polysaccharides, epoxy resins, silicone resins and/or polycondensation products of carbonyl compounds and NH group-containing compounds. Melamine-urea-formaldehyde microcapsules or melamine-formaldehyde microcapsules or urea-formaldehyde microcapsules can be preferably used, for example. Particularly preferred are microcapsules based on melamine-formaldehyde resins.

The general approach to producing microcapsules as such has long been known to a person skilled in the art. Particularly suitable methods for producing microcapsules are described in principle in U.S. Pat. Nos. 3,516,941, 3,415,758 or EP 0 026 914 A1, for example. The document mentioned last describes, for example, producing microcapsules by acid-induced condensation of melamine-formaldehyde precondensates and/or the C₁₋₄ alkyl ethers thereof in water, in which the hydrophobic material forming the capsule core is dispersed, in the presence of a protective colloid.

The core of the microcapsules contains at least one odor-neutralizing agent, as defined above.

In various embodiments, the core of the microcapsules further comprises at least one silicic acid ester of the general formula (III):

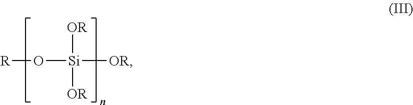

wherein, in the compounds according to formula (III), all the instances of R are selected, in each case independently of one another, from the group consisting of H, straight-chain or branched, saturated or unsaturated, substituted or unsubstituted C₁₋₆ hydrocarbon groups and fragrance alcohol groups; and n represents values from the range 2 to 100.

In various embodiments, the core of the microcapsule can also contain the compounds of formula (III) without containing a compound of formula (I) or (II). In such embodiments, the statements made above for the compounds of formulas (I) and (II) also apply to those of formula (III).

The compounds of the general formula (III) contain fragrance alcohols which have been reacted with silicic acids and their derivatives.

Oligosilicic acid esters of lower alcohols are commercially available, with methanol, ethanol, n-propanol, isopropanol, n-butanol, isobutanol and tert-butanol usually being used for esterification. The preparation of oligosilicic acid esters that have not been completely transesterified with fragrance alcohols leads to silicic acid ester mixtures in which a portion of the groups R is selected from the group methyl, ethyl, n-propyl, iso-propyl, n-butyl, iso-butyl and tert-butyl.

In various embodiments, a portion of the groups R, preferably at least 5 mol. % of the groups R, is selected from the group consisting of methyl, ethyl, n-propyl, iso-propyl, n-butyl, iso-butyl and tert-butyl.

Oligosilicic acid esters may contain at least one group R from the group of fragrance alcohol groups.

The compounds mentioned can be prepared by simple transesterification of oligosilicic acid esters of lower alcohols with fragrance alcohols, it being possible to use both individual fragrance alcohols and fragrance alcohol mixtures. Depending on the reaction time and reaction conditions, the lower alcohols are split off and the fragrance or biocide alcohols are bound, with the alcohols being more easily exchanged along Si—O—Si chains or rings than the terminal alcohols. The commercially available silicic acid esters are usually used as starting materials. The ethanol esters in particular should be mentioned here. The transesterification can be controlled exclusively by increasing the temperature and distilling off the volatile by-products. However, preference is given to using catalysts for the transesterification. These are usually Lewis acids, preferably aluminum tetraisopropylate, titanium tetraisopropylate, silicon tetrachloride or basic catalysts or also preparations such as aluminum oxide with potassium fluoride. The oligomeric silicic acid esters formed in this way then have at least some fragrance alcohol groups. However, the resulting esters usually also contain residues of lower alcohols. If small amounts of water or other hydrogen-acidic compounds are present during the preparation of the silicic acid esters, alcohol groups are also exchanged for OH groups. Accordingly, some of the silicic acid ester mixtures usually also contain hydrogen as R.

The completely transesterified oligosilicic acid esters may contain only a single group R, i.e., contain only a single fragrance alcohol.

The degrees of oligomerization “n” of the silicic acid esters are between 2 and 100, preferably between 2 and 50, more preferably between 2 and 20. In preferred compounds, n represents values between 2 and 15, preferably between 2 and 12 and in particular between 3 and 10, with the values 4, 5, 6, 7 and 8 being particularly preferred.

The term fragrance alcohols is understood to mean fragrances which have free hydroxyl groups that can be esterified, regardless of how the molecule is further structured. Thus, salicylic acid esters can also be used as fragrance alcohols. From the large group of fragrance alcohols, the following are mentioned by way of example and without limitation: 10-undecen-1-ol, 2,6-dimethylheptan-2-ol, 2-methylbutanol, 2-methylpentanol, 2-phenoxyethanol, 2-phenylpropanol, 2-tert-butycyclohexanol, 3,5,5-trimethylcyclohexanol, 3-hexanol, 3-methyl-5-phenylpentanol, 3-octanol, 3-phenyl-propanol, 4-heptenol, 4-isopropylcyclohexanol, 4-tert-butycyclohexanol, 6,8-dimethyl-2-nonanol, 6-nonen-1-ol, 9-decen-1-ol, α-methylbenzyl alcohol, α-terpineol, amyl salicylate, benzyl alcohol, benzyl salicylate, β-terpineol, butyl salicylate, citronellol, cyclohexyl salicylate, decanol, dihydromyrcenol, dimethyl benzyl carbinol, dimethyl heptanol, dimethyl octanol, ethyl salicylate, ethylvanillin, eugenol, farnesol, geraniol, heptanol, hexyl salicylate, isoborneol, isoeugenol, isopulegol, linalool, menthol, myrtenol, n-hexanol, nerol, nonanol, octanol, p-menthan-7-ol, phenylethyl alcohol, phenol, phenyl salicylate, tetrahydrogeraniol, tetrahydrolinalool, thymol, trans-2-cis-6-nonadicnol, trans-2-nonen-1-ol, trans-2-octenol, undecanol, vanillin, champiniol, hexenol and cinnamyl alcohol. Silicic acid esters may include those in which each R is selected, independently of one another, from the group of residues of the fragrance alcohols listed above and mixtures thereof. Further suitable biocide alcohols are 1,2-propylene glycol, glycerol, citric acid and its esters, lactic acid and its esters, salicylic acid and its esters, 2-benzyl-4-chlorophenol and 2,2′-methylene-bis-(6-bromo-4-chlorophenol).

In various embodiments, at least 10 mol. %, preferably at least 20 mol. % and particularly preferably even more than 40 mol. % of R selected from the group consisting of residues of the fragrance alcohols 10-undecen-1-ol, 2,6-dimethylheptan-2-ol, 2-methylbutanol, 2-methylpentanol, 2-phenoxyethanol, 2-phenylpropanol, 2-tert-butylcyclohexanol, 3,5,5-trimethylcyclohexanol, 3-hexanol, 3-methyl-5-phenylpentanol, 3-octanol, 3-phenylpropanol, 4-heptenol, 4-isopropylcyclohexanol, 4-tert-butylcyclohexanol, 6,8-dimethyl-2-nonanol, 6-nonen-1-ol, 9-decen-1-ol, α-methylbenzyl alcohol, α-terpineol, amyl salicylate, benzyl alcohol, benzyl salicylate, ß-terpineol, butyl salicylate, citronellol, cyclohexyl salicylate, decanol, dihydromyrcenol, dimethylbenzyl carbinol, dimethylheptanol, dimethyloctanol, ethyl salicylate, ethylvanillin, eugenol, farnesol, geraniol, heptanol, hexyl salicylate, isoborneol, isoeugenol, isopulegol, linalool, menthol, myrtenol, n-hexanol, nerol, nonanol, octanol, p-menthan-7-ol, phenylethyl alcohol, phenol, phenyl salicylate, tetrahydrogeraniol, tetrahydrolinalool, thymol, trans-2-cis-6-nonadicnol, trans-2-nonen-1-ol, trans-2-octenol, undecanol, vanillin, champiniol, hexenol, cinnamic alcohol and mixtures thereof.

Phenylethyl silicic acid esters, geranyl silicic acid esters, citronellyl silicic acid esters, cyanamyl silicic acid esters, hexenyl silicic acid esters, nonadienyl silicic acid esters, octenyl silicic acid esters or mixtures of two or more of these silicic acid esters are preferably used.

In various embodiments, the core may contain additional benefit agents. In some embodiments, such a further benefit agent is a perfume oil. However, it may be preferred that the core does not contain any other such benefit agent or perfume oil. However, such embodiments in which the core is free of further perfume oil include those microcapsules in which the core contains free perfume oil as a (spontaneous) release product from odor-neutralizing agents. In such a case, however, these are not deliberately added in free form, but originate from the odor-neutralizing agent used. However, these can also be contained in the agent in free form or in a separately encapsulated form.

As perfume oils, all substances and mixtures known for this purpose can be used. The terms “odorant(s),” “fragrances” and “perfume oil(s)” are used synonymously. The terms refer, in particular, to all substances or mixtures thereof that are perceived by humans and animals as having a smell, in particular perceived by humans as having a pleasant smell.

Perfumes, perfume oils, or perfume oil constituents may be used as fragrance components. Perfume oils or fragrances may be individual odorant compounds, such as synthetic products of the ester, ether, aldehyde, ketone, alcohol, and hydrocarbon types.

Fragrance compounds of the aldehyde type and of the ketone type have already been listed above.

Fragrance compounds of the alcohol type have likewise already been listed above as examples.

Fragrance compounds of the ester type are e.g., benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmacyclate.

Ethers include, for example, benzyl ethyl ether and Ambroxan. Hydrocarbons mainly include terpenes such as limonene and pinene.

Preferably, mixtures of different fragrances are used, which together produce an appealing fragrance note. Such a mixture of fragrances may also be referred to as perfume or perfume oil. Perfume oils of this kind may also contain natural fragrance mixtures, such as those obtainable from plant sources.

Fragrances of plant origin include essential oils such as angelica root oil, aniseed oil, arnica blossom oil, basil oil, bay oil, champaca blossom oil, citrus oil, abies alba oil, abies alba cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, jasmine oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, labdanum oil, lavender oil, lemon grass oil, lime blossom oil, lime oil, mandarin oil, melissa oil, mint oil, musk seed oil, muscatel oil, myrrh oil, clove oil, neroli oil, niaouli oil, olibanum oil, orange blossom oil, orange peel oil, oregano oil, palmarosa oil, patchouli oil, balsam Peru oil, petitgrain oil, pepper oil, peppermint oil, allspice oil, pine oil, rose oil, rosemary oil, sage oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil, and ambrettolide, Ambroxan, alpha-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, boisambrene forte, alpha-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methylanthranilic acid methyl ester, p-methylacetophenone, methyl chavicol, p-methylquinoline, methyl beta-naphthyl ketone, methyl n-nonyl acetaldehyde, methyl n-nonyl ketone, muscone, beta-naphthol ethyl ether, beta-naphthol methyl ether, nerol, n-nonyl aldehyde, nonyl alcohol, n-octyl aldehyde, p-oxy-acetophenone, pentadecanolide, beta-phenethyl alcohol, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, Troenan, gamma-undecalactone, vanillin, veratraldehyde, cinnamaldehyde, cinnamyl alcohol, cinnamic acid, cinnamic acid ethyl ester, cinnamic acid benzyl ester, diphenyl oxide, limonene, linalool, linalyl acetate and propionate, melusate, menthol, menthone, methyl-n-heptenone, pinene, phenylacetaldehyde, terpinyl acetate, citral, citronellal and mixtures thereof.

Mixtures of said substances may also be used.

If it is to be perceptible, an odorant must be volatile, and, in addition to the nature of the functional groups and the structure of the chemical compound, the molar mass also plays an important role. Therefore, most odorants have molar masses of up to approximately 200 daltons, while molar masses of 300 daltons and above are something of an exception. Due to the differing volatility of odorants, the odor of a perfume or fragrance composed of multiple odorants varies over the course of evaporation, the odor impressions being divided into “top note,” “middle note or body” and “end note or dry out.” Analogously to the description in the international patent publication WO 2016/200761 A2, the top, middle and end notes can be classified on the basis of their vapor pressure (determinable by means of the test methods described in WO 2016/200761) as follows:

Top notes: vapor pressure at 25° C.: >0.0133 kPa
Middle notes: vapor pressure at 25° C.: 0.0133 to 0.000133 kPa
End note: vapor pressure at 25° C.: <0.000133 kPa

Examples of adherent odorants that can be used are essential oils such as angelica root oil, aniseed oil, arnica blossom oil, basil oil, bay oil, bergamot oil, champaca blossom oil, abies alba oil, abies alba cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, melissa oil, musk seed oil, myrrh oil, clove oil, neroli oil, niaouli oil, olibanum oil, orange oil, oregano oil, palmarosa oil, patchouli oil, balsam Peru oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil, and cypress oil.

Higher-boiling or solid odorants of natural or synthetic origin include, for example: ambrettolide, α-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methylanthranilic acid methyl ester, p-methylacetophenone, methylchavicol, p-methylquinoline, methyl-o-naphthyl ketone, methyl n-nonyl acetaldehyde, methyl n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratraldehyde, cinnamaldehyde, cinnamyl alcohol, cinnamic acid, cinnamic acid ethyl ester and cinnamic acid benzyl ester

More volatile odorants include in particular lower-boiling odorants of natural or synthetic origin, which may be used alone or in mixtures. Examples of more volatile odorants are alkyl isothiocyanates (alkyl mustard oils), butanedione, limonene, linalool, linayl acetate and propionate, menthol, menthone, methyl-n-heptenone, phellandrene, phenylacetaldehyde, terpinyl acetate, citral and citronellal.

Odorant compounds of the aldehyde type that can preferably be used are hydroxycitronellal (CAS 107-75-5), Helional (CAS 1205-17-0), citral (5392-40-5), bourgeonal (18127-01-0), Triplal (CAS 27939-60-2), Ligustral (CAS 68039-48-5), vertocitral (CAS 68039-49-6), Florhydral (CAS 125109-85-5), citronellal (CAS 106-23-0), citronellyloxyacetaldehyde (CAS 7492-67-3).

In addition to or as an alternative to the above-mentioned odorants, it is also possible to use the odorants described in WO 2016/200761 A2, in particular the odorants mentioned in tables 1, 2 and 3, and the modulators listed in tables 4a and 4b. The whole of this publication is incorporated herein by way of reference.

Suitable perfume oils can also be contained as a constituent of the composition in the form of a perfume oil preparation and for example comprise at least one further active substance in oil form. Suitable active substances in oil form in this context are those which are suitable for washing, cleaning, care and/or finishing purposes, in particular
a) textile care substances, such as preferably silicone oils, and/or
b) skin care substances, such as preferably vitamin E, natural oils and/or cosmetic oils.

Skin care active substances are all those active substances which give the skin a sensory and/or cosmetic advantage. Skin care active substances are preferably selected from the following substances:
a) waxes such as carnauba, spermaceti, beeswax, lanolin and/or derivatives thereof and others.
b) hydrophobic plant extracts
c) hydrocarbons such as squalene and/or squalane
d) higher fatty acids, preferably those having at least 12 carbon atoms, for example lauric acid, stearic acid, behenic acid, myristic acid, palmitic acid, oleic acid, linoleic acid, linolenic acid, isostearic acid and/or polyunsaturated fatty acids and others.
e) higher fatty alcohols, preferably those having at least 12 carbon atoms, for example lauryl alcohol, cetyl alcohol, stearyl alcohol, oleyl alcohol, behenyl alcohol, cholesterol and/or 2-hexadecanol and others.
f) esters, preferably such as cetyloctanoate, lauryl lactate, myristyl lactate, cetyl lactate, isopropyl myristate, myristyl myristate, isopropyl palmitate, isopropyl adipate, butyl stearate, decyl oleate, cholesteryl isostearate, glycerol monostearate, glyceryl distearate, glycerol tristearate, alkyl lactate, alkyl citrate and/or alkyl tartrate and others.
g) lipids such as cholesterol, ceramides and/or sucrose esters and others.
h) vitamins such as vitamins A, C and E, vitamin alkyl esters, including vitamin C alkyl esters and others.
i) sunscreens
j) phospholipids
k) derivatives of alpha hydroxy acids
l) germicides for cosmetic use, both synthetic such as salicylic acid and/or others and natural such as neem oil and/or others.
m) silicones
n) natural oils, e.g., almond oil
   and mixtures of any of the components listed above.

In various embodiments, the core of the microcapsules can, in addition to the at least one odor-neutralizing agent and optionally at least one silicic acid ester compound, also include in particular at least one fragrance aldehyde and/or fragrance ketone, each preferably selected from the group of the aforementioned fragrance aldehydes and fragrance ketones. However, it can also be preferable for the core of the microcapsules to be free of such additional fragrances and for these to be formulated separately in the agent.

In preferred embodiments, the microcapsule, in particular the core of the microcapsule, in addition to the at least one odor-neutralizing agent, i.e., at least one 1-aza-3,7-dioxabicyclo[3.3.0]octane compound of the general formula (II) and/or at least one 2-amino-1,3-propanediol compound of the general formula (I), and optionally at least one silicic acid ester compound of formula (III) and/or optionally at least one compound of formula (IV), is free of additional free fragrances, i.e., the microcapsule, in particular the core of the microcapsule, contains no additional free fragrances/odorants as defined above.

“Additional free fragrances” refer to further free fragrances that, in addition to the compounds of formulas (I) and/or (II) and optionally the compounds of formulas (III) and/or (IV), may be contained in the microcapsules, in particular in the core of the microcapsules. This does not include fragrance aldehydes, fragrance ketones and/or fragrance alcohols which can be released from the compounds of formulas (II), (III) and/or (IV). The microcapsules, in particular the cores of the microcapsules, are free of additional free fragrances in addition to the compounds of formulas (I) and/or (II) and, optionally, the compounds of formulas (III) and/or (IV), apart from the free fragrance aldehydes, fragrance ketones and/or fragrance alcohols which can be released from the compounds of formulas (II), (III) and/or (IV).

In various embodiments, suitable microcapsules can further contain plant extracts as an active substance. These extracts are typically prepared by extraction of the entire plant. It may also be preferable in some cases, however, to prepare the extracts solely from the flowers and/or leaves of the plant.

Extracts from green tea, oak bark, stinging nettle, witch hazel, hops, henna, chamomile, burdock root, horsetail, whitethorn, lime blossom, almond, aloe vera, spruce needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, meristem, ginseng and ginger root are particularly suitable. Extracts of aloe vera are particularly preferred.

Water, alcohols, and mixtures thereof can be used as extracting agents for preparing the mentioned plant extracts. Of the alcohols, low alcohols such as ethanol and isopropanol, but in particular polyhydric alcohols such as ethylene glycol and propylene glycol, are preferred, both as the sole extracting agent and in a mixture with water. Plant extracts based on water/propylene glycol in the ratio of from 1:10 to 10:1 have been found to be particularly suitable.

The plant extracts can be used both in pure form and in diluted form. If they are used in diluted form, they typically contain approximately 2 to 80 wt. % active substance and the extracting agent or extracting agent mixture used for their extraction as the solvent.

It may also be preferable to use a plurality of, in particular two, different plant extracts as the active substance.

In various embodiments, the microcapsules described above accordingly contain at least one odor-neutralizing agent and optionally at least one silicic acid ester of the general formula (III), which can also be a constituent of a benefit agent composition, i.e., can be contained in combination with further fragrances or odorants, for example perfume oils, as described above. In some embodiments, the at least one odor-neutralizing agent and optionally further benefit agents and/or at least one silicic acid ester, as defined above, is contained in the microcapsules in an amount of from approximately 30 to approximately 95 wt. %, preferably at least 40 to approximately 90 wt. % more preferably at least 50 to approximately 90 wt. %, in particular at least 60 to approximately 90 wt. %, in each case based on the total weight of the microcapsules.

Suitable microcapsules can also be contained in the composition in the form of a (capsule) slurry, i.e., a suspension of the capsules in a liquid medium.

The term “slurry” refers to a, typically aqueous, suspension of microcapsules, as defined above. The liquid medium preferably consists predominantly of water, i.e. in an amount of more than 50 wt. %, but can also completely consist of water, i.e. in an amount of 100%. The slurry is preferably pourable, i.e., it can be poured out of a vessel by tilting the vessel. A pourable slurry is understood to mean, in particular, a capsule/liquid mixture which has a viscosity of less than 10-10⁴ mPas (Brookfield rotational viscometer; spindle 2, 20 rpm) at the working temperature, preferably at a maximum of 40° C., in particular at a maximum of 20° C.

The slurry may contain further auxiliaries, for example those which ensure a certain durability or stability. Frequently used auxiliaries include, for example, surfactants, in particular anionic and/or non-ionic surfactants. Corresponding capsule slurries are commercially available and known to a person skilled in the art per se.

In various embodiments, the capsules described above are contained in the slurry in an amount of from 5 to 80 wt. %, preferably 10 to 70 wt. %, in particular 20 to 60 wt. %, for example 30 to 40 wt. %.

In various embodiments, the microcapsules described above are contained in the composition in an amount of from approximately 0.01 to 50 wt. %, preferably approximately 0.1 to 25 wt. %, in particular 0.1 to 10 wt. %, in each case based on the total weight of said composition.

In various embodiments, the microcapsules described above are in particular contained in the composition in an amount of from approximately 0.01 to 50 wt. %, preferably approximately 0.1 to 25 wt. %, in particular 0.1 to 10 wt. %, in each case based on the total weight of said composition, the composition being a fabric softener composition or fabric finisher composition. In this embodiment, the microcapsules are preferably in the form of a capsule slurry.

In various embodiments, the microcapsules described above are in particular contained in the composition in an amount of from approximately 0.01 to 50 wt. %, preferably approximately 0.1 to 25 wt. %, in particular 0.1 to 10 wt. %, in each case based on the total weight of said composition, the composition being a liquid washing agent composition. In this embodiment, the microcapsules are preferably in the form of a capsule slurry.

In various embodiments, the at least one odor-neutralizing agent is present in the composition in an amount of from approximately 0.1 to approximately 40 wt. %, preferably approximately 0.1 to approximately 20 wt. %, based on the total weight of said composition.

In various embodiments, the at least one silicic acid ester as defined above is contained in the composition in an amount of from approximately 0.1 to approximately 40 wt. %, preferably approximately 0.1 to approximately 20 wt. %, in each case based on the total weight of said composition.

In various embodiments, the composition also comprises at least one further benefit agent, i.e., in addition to the aforementioned benefit agents, for example perfume oils, which are present in encapsulated form, at least one further non-encapsulated benefit agent. Suitable further benefit agents are not subject to any restrictions and can be selected from benefit agents known in the prior art and suitable for use in washing, cleaning and treatment methods, such as fragrances and odorants, fragrance precursor compounds, skin care active substances, etc.

In various embodiments, the composition, as described above, is a washing agent additive, cleaning agent additive or treatment agent additive. For example, without limitation, such an additive may be in the form of a liquid or solid composition.

“Solid,” as used herein, denotes a powder composition, granulate composition, extrudate composition or compact composition.

“Liquid,” denotes all flowable compositions (at 20° C., 1.013 bar), including gels and paste-like compositions, and also non-Newtonian liquids that have a yield point.

An agent may include a composition as described herein, which can be a washing agent, cleaning agent or treatment additive according to some embodiments. Correspondingly, in some embodiments, the agent is a washing agent, cleaning agent or treatment agent, for example a cosmetic product, washing agent, cleaning agent or textile treatment agent.

Agents are suitable for washing, cleaning, conditioning, caring for and/or dyeing hard or soft surfaces. Within the meaning of this application, agents can consequently be washing agents, cleaning agents, aftertreatment agents and/or cosmetic agents. Agents also include agents for the air care sector, i.e., they are suitable for improving room air or for combating malodors.

Within the meaning of this application, hard surfaces are windows, mirrors and other glass surfaces, surfaces made of ceramic, plastics material, metal or wood and lacquered wood, which are found in the household and commercial sectors, such as bathroom ceramics, cooking and dining utensils, kitchen surfaces or floors. Within the meaning of this application, soft surfaces are textile sheet materials, skin and hair.

Within the meaning of this application, agents for washing hard or soft surfaces are textile washing agents, for example formulations in the form of powders, granules, beads, tablets, pastes, gels, wipes, bars or liquids.

Within the meaning of this application, agents for cleaning hard or soft surfaces include all cleaners for hard or soft surfaces, in particular dishwashing detergents, all-purpose cleaners, toilet cleaners, sanitary cleaners and glass cleaners, toothpastes, skin washing agents, such as shower gels, or hair washing agents.

Within the meaning of this application, agents for conditioning hard or soft surfaces are fabric softeners, rim blocks, conditioning wipes for use in tumble dryers, hygiene rinsers, deodorants, antiperspirants, hair conditioners, styling agents and/or hair setting agents.

Within the meaning of this application, agents for caring for hard or soft surfaces are textile care products, hair care agents or skin treatment agents, such as creams, lotions or gels.

Within the meaning of this application, agents for dyeing hard or soft surfaces are hair dyeing and hair toning agents and agents for lightening keratin fibers.

Air care agents, in other words room air treatment (enhancement) agents, include, but are not limited to, room sprays, textile sprays, and fragrance delivery systems.

In a preferred embodiment, the surface is a textile surface. When the surface is a textile surface, it is particularly preferable for the agent for washing, cleaning, conditioning, caring for and/or dyeing hard or soft surfaces to be a washing agent, cleaning agent or post-treatment agent.

In a further embodiment, the surface is a body part, in particular skin and/or hair. When the surface is a body part, in particular skin and/or hair, it is preferable for the agent for washing, cleaning, conditioning, caring for and/or dyeing hard or soft surfaces to be a cosmetic composition.

An agent comprises at least one type of a composition as defined and described herein. In preferred embodiments, such an agent contains at least one composition in an amount of up to approximately 30 wt. %, preferably up to approximately 20 wt. %, more preferably up to approximately 15 wt. %, in particular up to approximately 10 wt. %, for example up to at 1, 2, 3, 4, 5, 6, 7, 8, 9 or 10 wt. %.

In addition to the composition described herein, an agent can further contain conventional ingredients and further ingredients known per se to a person skilled in the art, for example at least one or preferably more substances from the group of enzymes, surfactants, bleaching agents, complexing agents, builders, electrolytes, non-aqueous solvents, pH adjusters, further fragrances, further fragrance carriers, fluorescing agents, dyes, hydrotropic substances, suds suppressors, silicone oils, anti-redeposition agents, graying inhibitors, anti-shrink agents, anti-crease agents, dye transfer inhibitors, antimicrobial active ingredients, germicides, fungicides, antioxidants, corrosion inhibitors, antistatic agents, bittering agents, ironing aids, repellents and impregnating agents, anti-swelling and anti-slip agents, softening components and UV absorbers.

A composition or an agent may be used for reducing malodors, and to a method for treating a surface, for treating room air, or for washing and/or caring for textiles, characterized in that a composition or an agent, as described above, is used in at least one method step.

All aspects, objects, and embodiments described for the compositions are also applicable to the above-mentioned methods, uses, and agents containing these compositions. Therefore, reference is expressly made at this point to the disclosure at the corresponding point where it is indicated that this disclosure also applies to the above-described methods and uses.

The invention is described in the following with reference to examples, but is not limited to these examples.

### EXAMPLES

Examples of formulations and comparison formulations are listed in the following. Corresponding formulations were tested for their odor quality (i.e., intensity of malodor neutralization) in performance test series. The corresponding results are summarized in a table (scale 1-10, where 1 means very weak and 10 means very strong).

### Example 1

Product: universal washing agent
Product dosage: 50 ml
Washing conditions: main wash at 40° C.
Malodor: sweat
Textile type: polyester

TABLE 1

| | Intensity of malodor | | | | | |
|---|---|---|---|---|---|---|
| | after | after | after | after | after | after |
| Formulation | 0 h | 2 h | 4 h | 6 h | 24 h | 48 h |
| Non-perfumed formulation | 10 | 9 | 7 | 6 | 5 | 3 |
| 1% perfume containing 5% 1H, 3H, 5H-oxazolo[3,4-c] | 9 | 6 | 5 | 4 | 4 | 3 |
| oxazole, 3,5-diheptyldihydro-7a-methyl-—non-encapsulated | | | | | | |
| 1% perfume containing 5% 1H, 3H, 5H-oxazolo[3,4-c] | 7 | 4 | 3 | 2 | 2 | 2 |
| oxazole, 3,5-diheptyldihydro-7a-methyl-—encapsulated | | | | | | |

### Example 2

Product: fabric softener
Product dosage: 30 ml
Washing conditions: main wash at 40° C.
Malodor: sweat
Textile type: cotton

TABLE 2

| | Intensity of malodor | | | | | |
|---|---|---|---|---|---|---|
| | after | after | after | after | after | after |
| Formulation | 0 h | 2 h | 4 h | 6 h | 24 h | 48 h |
| Non-perfumed formulation | 9 | 8 | 6 | 5 | 4 | 3 |
| 1.5% perfume containing 5% 1H, 3H, 5H-oxazolo[3,4-c] | 8 | 7 | 5 | 3.5 | 3 | 2.5 |
| oxazole, 3,5-diheptyldihydro-7a-methyl-—non-encapsulated | | | | | | |
| 1.5% perfume containing 5% 1H, 3H, 5H-oxazolo[3,4-c] | 6 | 4 | 2 | 2 | 1.5 | 1.5 |
| oxazole, 3,5-diheptyldihydro-7a-methyl-—encapsulated | | | | | | |

### Example 3

Product: color catcher sheet
Product dosage: 1 sheet
Washing conditions: main wash at 40° C.
Malodor: sweat
Textile type: cotton

TABLE 3

| | Intensity of malodor | | | | | |
|---|---|---|---|---|---|---|
| | after | after | after | after | after | after |
| Formulation | 0 h | 2 h | 4 h | 6 h | 24 h | 48 h |
| Non-perfumed formulation | 10 | 9 | 9 | 8 | 7 | 5 |
| 4% perfume containing 5% 1H, 3H, 5H-oxazolo[3,4-c] | 8 | 7 | 5 | 4 | 3.5 | 3 |
| oxazole, 3,5-diheptyldihydro-7a-methyl-—non-encapsulated | | | | | | |
| 4% perfume containing 5% 1H, 3H, 5H-oxazolo[3,4-c] | 5 | 4 | 3 | 3 | 2 | 1.5 |
| oxazole, 3,5-diheptyldihydro-7a-methyl-—encapsulated | | | | | | |

### Example 4

Product: universal washing agent
Product dosage: 50 ml
Washing conditions: main wash at 40° C.
Malodor: mold
Textile type: polyester

TABLE 4

| | Intensity of malodor | | | | | |
|---|---|---|---|---|---|---|
| | after | after | after | after | after | after |
| Formulation | 0 h | 2 h | 4 h | 6 h | 24 h | 48 h |
| Non-perfumed formulation | 10 | 9 | 8 | 8 | 5 | 3 |
| 1% perfume containing 5% 1H, 3H, 5H-oxazolo[3,4-c] | 8 | 6 | 4 | 4 | 3 | 3 |
| oxazole, 3,5-diheptyldihydro-7a-methyl-—non-encapsulated | | | | | | |
| 1% perfume containing 5% 1H, 3H, 5H-oxazolo[3,4-c] | 7 | 6 | 3 | 3 | 2 | 2 |
| oxazole, 3,5-diheptyldihydro-7a-methyl-—encapsulated | | | | | | |

### Example 5

Product: fabric finisher
Product dosage: 30 g
Washing conditions: main wash at 40° C.
Malodor: sweat
Textile type: cotton

TABLE 5

| | | Intensity of malodor | | | |
|---|---|---|---|---|---|
| | | after | | | |
| | | 0 h | | | |
| | | (directly | | | |
| | | after | | | after |
| | | application | | | 4 h |
| | | of the | after | after | after |
| Formulation | Dosage | malodor) | 2 h | 4 h | rubbing |
| Odor neutralizer 1 - non- | 0.1% | 7 | 6.0 | 5.0 | 4.0 |
| encapsulated | | | | | |
| Odor neutralizer 2 - non- | 0.1% | 8 | 7.0 | 5.5 | 4.0 |
| encapsulated | | | | | |
| Odor neutralizer 3 - non- | 0.1% | 8 | 7.0 | 6.0 | 6.0 |
| encapsulated | | | | | |
| MF capsule slurry with | 0.3% | 7.5 | 5.0 | 4.0 | 2.0 |
| odor-neutralizing agent 1 | | | | | |
| (contains 34% odor | | | | | |
| neutralizer 1) | | | | | |
| MF capsule slurry with | 0.3% | 8 | 6.0 | 4.0 | 2.5 |
| odor-neutralizer 2 | | | | | |
| (contains 34% odor | | | | | |
| neutralizer 2) | | | | | |
| MF capsule slurry with | 0.3% | 8 | 6.5 | 5.0 | 4.0 |
| odor neutralizer 3 | | | | | |
| (contains 34% odor | | | | | |
| neutralizer 3) | | | | | |

Odor neutralizer 1: 3,5-bis(2,4-dimethylcyclohex-3-en-1-yl)dihydro-1H-[1,3]oxazolo[3,4-c][1,3]oxazole
Odor neutralizer 2: 1H,3H,5H-oxazolo[3,4-c]oxazole, 3,5-diheptyldihydro-7a-methyl-
Odor neutralizer 3: Zinc ricinoleate

1. A composition comprising:
microcapsules comprising at least one core and one shell; wherein the at least one core contains at least one odor-neutralizing agent selected from:
a) a 2-amino-1,3-propanediol compound of the general formula (I) 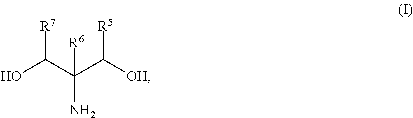
wherein, in the compounds according to formula (I), R⁵, R⁶, and R⁷, in each case independently of one another, represent H or hydrocarbon groups;
b) a 1-aza-3,7-dioxabicyclo[3.3.0]octane compound of the general formula (II) 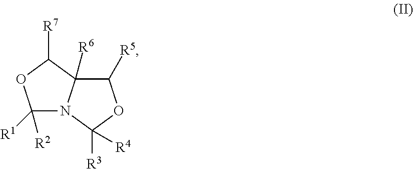
wherein, in the compounds according to formula (II), R¹, R², R³, and R⁴, in each case independently of one another, represent groups which, in a compound of the general formula R¹—C(═O)—R² or R³—C(═O)—R⁴, produce a fragrance aldehyde having at least 6 carbon atoms or a fragrance ketone having at least 6 carbon atoms, and R⁵, R⁶, and R⁷, in each case independently of one another, represent H or a hydrocarbon group that is acyclic or cyclic, substituted or unsubstituted, branched or unbranched, saturated or unsaturated; or
c) combinations of compounds a) and b).

2. The composition according to claim 1, wherein the microcapsules are free of additional free fragrance.

3. The composition according to claim 1, wherein in the compounds of formula (II),
R², R⁴, R⁵, R⁶, and R⁷ represent H and R¹ and R³ represent, in each case independently, a C₅₋₂₄ hydrocarbon group; or
R⁵ and R⁷, in each case independently of one another, represent hydrogen or a C₁₋₆ hydrocarbon group which is optionally substituted; and/or
R⁶ represents H, a methyl, ethyl, or hydroxymethyl group; and/or
R² and R⁴ each represent H; and/or
R², R⁴, R⁵ and R⁷ each represent H, R⁶ represents H, a methyl, ethyl, or hydroxymethyl group; R¹ and R³ represent, in each case independently of one another, a C₆₋₂₄ hydrocarbon group, where the hydrocarbon group is acyclic or cyclic, substituted or unsubstituted, branched or unbranched, saturated or unsaturated; or
R², R⁴, R⁵, R⁶, and R⁷ represent H; and R¹ and R³ represent, in each case independently, a C₅₋₂₄ hydrocarbon group.

4. The composition according to claim 1, wherein
in the compounds of formula (II), R¹, R², R³, and R⁴ in a compound of the general formula R¹—C(═O)—R² or R³—C(═O)—R⁴ produce a fragrance aldehyde or a fragrance ketone, the fragrance aldehyde being selected from the group consisting of adoxal (2,6,10-trimethyl-9-undecenal), anisaldehyde (4-methoxybenzaldehyde), cymene (3-(4-isopropylphenyl)-2-methylpropanal), ethylvanillin, Florhydral (3-(3-isopropylphenyl)butanal), helional (3-(3,4-methylenedioxyphenyl)-2-methylpropanal), heliotropin, hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenealdehyde, vanillin, 2,6,10-trimethyl-9-undecenal, 3-dodecen-1-al, alpha-n-amylcinnamaldehyde, melonal (2,6-dimethyl-5-heptenal), 2,4-dimethyl-3-cyclohexene-1-carboxaldehyde (Triplal), 4-methoxybenzaldehyde, benzaldehyde, 3-(4-tert-butylphenyl)-propanal, 2-methyl-3-(para-methoxyphenyl)propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, 3-ethoxy-4-hydroxybenzaldehyde, para-ethyl-alpha,alpha-dimethylhydrocinnamaldehyde, alpha-methyl-3,4-(methylenedioxy)-hydrocinnamaldehyde, 3,4-methylenedioxybenzaldehyde, alpha-n-hexylcinnamaldehyde, m-cymene-7-carboxaldehyde, alpha-methyl-phenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexenecarboxaldehyde, 1-dodecanal, 2,4-dimethyl-cyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanindan-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexenecarboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanindan-1-carboxaldehyde, 2-methyloctanal, alpha-methyl-4-(1-methylethyl)benzeneacetaldehyde, 6,6-dimethyl-2-norpinene-2-propionaldehyde, para-methylphenoxyacetaldehyde, 2-methyl-3-phenyl-2-propen-1-al, 3,5,5-trimethylhexanal, hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonylacetaldehyde, hexanal, and trans-2-hexenal;
and/or the fragrance ketone being selected from the group consisting of methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, Iso-E-Super (1-(1,2,3,4,5,6,7,8-octahydro-2,3,8,8-tetramethyl-2-naphthalenyl)-ethan-1-one (and isomers)), methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyldecahydro-2-naphthone, dimethyloctenone, frescomenthe (2-butan-2-yl-cyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menthen-6(2)-yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl (4-(1,3-benzodioxol-5-yl)butan-2-one), Hexalone (1-(2,6,6-trimethyl-2-cyclohexen-1-yl)-1,6-heptadien-3-one), Isocyclemone E (2-acetonaphthone-1,2,3,4,5,6,7,8-octahydro-2,3,8,8-tetramethyl), methyl nonylketone, methylcyclocitrone, methyl lavender ketone, Orivone (4-tert-amyl-cyclohexanone), 4-tert-butylcyclohexanone, Delphone (2-pentyl-cyclopentanone), muscone (CAS 541-91-3), Neobutenone (1-(5,5-dimethyl-1-cyclohexenyl)pent-4-en-1-one), plicatone (CAS 41724-19-0), Veloutone (2,2,5-trimethyl-5-pentylcyclopentan-1-one), 2,4,4,7-tetramethyl-oct-6-en-3-one, and tetramerane (6,10-dimethylundecen-2-one); and/or
the core of the microcapsules further comprising at least one fragrance aldehyde and/or fragrance ketone.

5. The composition according to claim 1, wherein the core of the microcapsules further comprises at least one silicic acid ester of the general formula (III): 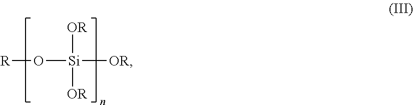
wherein, in the compounds according to formula (III), all R are selected, in each case independently of one another, from the group consisting of H, straight-chain or branched, saturated or unsaturated, substituted or unsubstituted C₁-C₆ hydrocarbon groups and fragrance alcohol groups; and n represents values ranging from 2 to 100.

6. The composition according to claim 5, wherein, in the compounds of formula (III),
a portion of the R groups is selected from the group consisting of methyl, ethyl, n-propyl, iso-propyl, n-butyl, iso-butyl, and tert-butyl; and/or
at least 10 mol. %, are selected from the group consisting of the residues of the fragrance alcohols 10-undecen-1-ol, 2,6-dimethylheptan-2-ol, 2-methylbutanol, 2-methylpentanol, 2-phenoxyethanol, 2-phenylpropanol, 2-tert-butylcyclohexanol, 3,5,5-trimethylcyclohexanol, 3-hexanol, 3-methyl-5-phenylpentanol, 3-octanol, 3-phenylpropanol, 4-heptenol, 4-isopropylcyclohexanol, 4-tert-butylcyclohexanol, 6,8-dimethyl-2-nonanol, 6-nonen-1-ol, 9-decen-1-ol, α-methyl benzyl alcohol, α-terpineol, amyl salicylate, benzyl alcohol, benzyl salicylate, ß-terpineol, butyl salicylate, citronellol, cyclohexyl salicylate, decanol, dihydromyrcenol, dimethyl benzyl carbinol, dimethyl heptanol, dimethyl octanol, ethyl salicylate, ethylvanillin, eugenol, farnesol, geraniol, heptanol, hexyl salicylate, isobornene, isoeugenol, isopulegol, linalool, menthol, myrtenol, n-hexanol, nerol, nonanol, octanol, p-menthan-7-ol, phenylethyl alcohol, phenol, phenyl salicylate, tetrahydrogeraniol, tetrahydrolinalool, thymol, trans-2-cis-6-nonadicnol, trans-2-nonen-1-ol, trans-2-octenol, undecanol, vanillin, champiniol, hexenol, cinnamic alcohol, and mixtures thereof; and/or
n represents values ranging from 2 to 50.

7. The composition according to claim 1, wherein:
the composition is a washing, cleaning, or treatment agent additive;
the composition contains at least one further benefit agent;
the at least one odor-neutralizing agent is present in the composition in an amount ranging from approximately 0.1 to approximately 40 wt. % based on the total weight of said composition;
the at least one silicic acid ester is contained in the composition in an amount ranging from approximately 0.1 to approximately 40 wt. % in each case based on the total weight of said composition; and
combinations thereof.

8. The composition according to claim 1, wherein:
the microcapsules are present in an amount ranging from approximately 0.01 to 50 wt. % in each case based on the total weight of the composition;
the microcapsules are contained in the form of a capsule slurry; and
combinations thereof.

9. An agent comprising at least one composition according to claim 1, wherein the agent is a washing agent, cleaning agent, or treatment agent.

10. The agent according to claim 9, wherein the at least one composition according to claim 1 is present in the agent in an amount of up to approximately 30 wt. %.

11. The agent according to claim 9, further comprising at least one component selected from enzymes, surfactants, bleaching agents, complexing agents, builders, electrolytes, non-aqueous solvents, pH adjusters, further fragrances, further fragrance carriers, fluorescing agents, dyes, hydrotropic substances, suds suppressors, silicone oils, anti-redeposition agents, graying inhibitors, shrinkage inhibitors, anti-crease agents, dye transfer inhibitors, antimicrobial active ingredients, germicides, fungicides, antioxidants, corrosion inhibitors, antistatic agents, bittering agents, ironing aids, repellents and impregnating agents, swelling and non-slip agents, softening components, UV absorbers, and combinations thereof.

12. (canceled)

13. A method comprising: applying a composition according to claim 1 to a surface, room air, a textile, or combinations thereof.

14. The composition according to claim 1, wherein R⁵ and R⁷, in each case independently of one another, represent a C₁₋₃ hydrocarbon group.

15. The composition according to claim 1, wherein R⁵ and R⁷, in each case independently of one another, are each selected from hydrogen, methyl, or an ethyl group.

16. The composition according to claim 1, wherein R¹ and R³ represent, in each case independently of one another, a C₇₋₂₄ hydrocarbon group.

17. The composition according to claim 4, wherein the at least one fragrance aldehyde of the core of the microcapsules is selected from the group consisting of adoxal (2,6,10-trimethyl-9-undecenal), anisaldehyde (4-methoxybenzaldehyde), cymene (3-(4-isopropylphenyl)-2-methylpropanal), ethylvanillin, Florhydral (3-(3-isopropylphenyl)butanal), helional (3-(3,4-methylenedioxyphenyl)-2-methylpropanal), heliotropin, hydroxycitronellal, lauraldehyde, Lyral (3- and 4-(4-hydroxy-4-methylpentyl)-3-cyclohexene-1-carboxaldehyde), methylnonylacetaldehyde, Lilial (3-(4-tert-butylphenyl)-2-methylpropanal), phenylacetaldehyde, undecylenealdehyde, vanillin, 2,6,10-trimethyl-9-undecenal, 3-dodecen-1-al, alpha-n-amylcinnamaldehyde, melonal (2,6-dimethyl-5-heptenal), 2,4-dimethyl-3-cyclohexene-1-carboxaldehyde (Triplal), 4-methoxybenzaldehyde, benzaldehyde, 3-(4-tert-butylphenyl)-propanal, 2-methyl-3-(para-methoxyphenyl)propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, 3-ethoxy-4-hydroxybenzaldehyde, para-ethyl-alpha,alpha-dimethylhydrocinnamaldehyde, alpha-methyl-3,4-(methylenedioxy)-hydrocinnamaldehyde, 3,4-methylenedioxybenzaldehyde, alpha-n-hexylcinnamaldehyde, m-cymene-7-carboxaldehyde, alpha-methyl-phenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexenecarboxaldehyde, 1-dodecanal, 2,4-dimethyl-cyclohexene-3-carboxaldehyde, 4-(4-hydroxy-4-methylpentyl)-3-cyclohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanindan-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexenecarboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanindan-1-carboxaldehyde, 2-methyloctanal, alpha-methyl-4-(1-methylethyl)benzeneacetaldehyde, 6,6-dimethyl-2-norpinene-2-propionaldehyde, para-methylphenoxyacetaldehyde, 2-methyl-3-phenyl-2-propen-1-al, 3,5,5-trimethylhexanal, hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonylacetaldehyde, hexanal, and trans-2-hexenal;
and/or the fragrance ketone of the core of the microcapsules is selected from the group consisting of methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, Iso-E-Super (1-(1,2,3,4,5,6,7,8-octahydro-2,3,8,8-tetramethyl-2-naphthalenyl)-ethan-1-one (and isomers)), methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyldecahydro-2-naphthone, dimethyloctenone, frescomenthe (2-butan-2-yl-cyclohexan-1-one), 4-(1-ethoxyvinyl)-3,3,5,5-tetramethylcyclohexanone, methylheptenone, 2-(2-(4-methyl-3-cyclohexen-1-yl)propyl)cyclopentanone, 1-(p-menthen-6(2)-yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl (4-(1,3-benzodioxol-5-yl)butan-2-one), Hexalone (1-(2,6,6-trimethyl-2-cyclohexen-1-yl)-1,6-heptadien-3-one), Isocyclemone E (2-acetonaphthone-1,2,3,4,5,6,7,8-octahydro-2,3,8,8-tetramethyl), methyl nonylketone, methylcyclocitrone, methyl lavender ketone, Orivone (4-tert-amyl-cyclohexanone), 4-tert-butylcyclohexanone, Delphone (2-pentyl-cyclopentanone), muscone (CAS 541-91-3), Neobutenone (1-(5,5-dimethyl-1-cyclohexenyl)pent-4-en-1-one), plicatone (CAS 41724-19-0), Veloutone (2,2,5-trimethyl-5-pentylcyclopentan-1-one), 2,4,4,7-tetramethyl-oct-6-en-3-one, and tetramerane (6,10-dimethylundecen-2-one).

18. The composition according to claim 6, wherein the portion of the R groups is at least 5 mol. % that are selected from the group consisting of methyl, ethyl, n-propyl, iso-propyl, n-butyl, iso-butyl, and tert-butyl.

19. The composition according to claim 6, wherein at least 40 mol. % of the R groups are selected from the group consisting of the residues of the fragrance alcohols 10-undecen-1-ol, 2,6-dimethylheptan-2-ol, 2-methylbutanol, 2-methylpentanol, 2-phenoxyethanol, 2-phenylpropanol, 2-tert-butylcyclohexanol, 3,5,5-trimethylcyclohexanol, 3-hexanol, 3-methyl-5-phenylpentanol, 3-octanol, 3-phenylpropanol, 4-heptenol, 4-isopropylcyclohexanol, 4-tert-butylcyclohexanol, 6,8-dimethyl-2-nonanol, 6-nonen-1-ol, 9-decen-1-ol, α-methyl benzyl alcohol, α-terpineol, amyl salicylate, benzyl alcohol, benzyl salicylate, ß-terpineol, butyl salicylate, citronellol, cyclohexyl salicylate, decanol, dihydromyrcenol, dimethyl benzyl carbinol, dimethyl heptanol, dimethyl octanol, ethyl salicylate, ethylvanillin, eugenol, farnesol, geraniol, heptanol, hexyl salicylate, isobornene, isoeugenol, isopulegol, linalool, menthol, myrtenol, n-hexanol, nerol, nonanol, octanol, p-menthan-7-ol, phenylethyl alcohol, phenol, phenyl salicylate, tetrahydrogeraniol, tetrahydrolinalool, thymol, trans-2-cis-6-nonadicnol, trans-2-nonen-1-ol, trans-2-octenol, undecanol, vanillin, champiniol, hexenol, cinnamic alcohol, and mixtures thereof.

20. The composition according to claim 6, wherein n represents values ranging from 3 to 10.

21. The composition according to claim 8, wherein the microcapsules are present in an amount ranging from 0.1 to 10 wt. %, in each case based on the total weight of the composition.
